(19) United States
(12) Patent Application Publication
(10) Pub. No.: US 2021/0283308 A1
(43) Pub. Date: Sep. 16, 2021

(54) IMPLANTABLE DEVICE FOR RETAINING LIVE CELLS AND PROVIDING NUTRIENTS THERETO

(71) Applicants: City of Hope, Durate, CA (US); California Institute of Technology, Pasadena, CA (US)

(72) Inventors: Hirotake Komatsu, Temple City, CA (US); Colin Andrew Cook, Pasadena, CA (US); Yu-Chong Tai, Pasadena, CA (US); Kuang-Ming Shang, Pasadena, CA (US)

(21) Appl. No.: 17/199,844

(22) Filed: Mar. 12, 2021

Related U.S. Application Data
(60) Provisional application No. 62/989,204, filed on Mar. 13, 2020.

(51) Int. Cl. A61L 27/38; A61L 27/18; A61B 17/34; A61K 35/39; A61L 27/54; A61L 27/56

(52) U.S. Cl. CPC A61L 27/3804; A61L 27/3834; A61L 27/18; A61B 17/3468; A61K 35/39; A61L 27/54; A61L 27/56; A61B 17/3421

(57) ABSTRACT

An oxygen transporter device configured to transport O2 from an ambient air location into or toward a subcutaneous site. The device includes an absorption component, a cannula, and a discharge component configured to be implanted in a subcutaneous location while the absorption component is not subcutaneous. The cannula can extend through or across the skin of a patient. The discharge component can includes one or more multi-layered, flexible mesh sheets, which are coupled to cells.

### CROSS REFERENCE TO RELATED APPLICATION

This application claims priority to U.S. Provisional Patent Application No. 62/989,204 filed Mar. 13, 2020, entitled “IMPLANTABLE DEVICE FOR RETAINING LIVE CELLS AND PROVIDING NUTRIENTS THERETO”, the contents of which is hereby incorporated by reference in its entirety and for all purposes.

### BACKGROUND

Diabetes is a group of widespread diseases in which there are high blood sugar levels over a prolonged period. If left untreated, diabetes can cause many complications. Acute complications can include diabetic ketoacidosis, nonketotic hyperosmolar coma, or death. Serious long-term complications include heart disease, stroke, chronic kidney failure, foot ulcers, and damage to the eyes. Diabetes is due to either cells in the pancreas not producing insulin (type-I diabetes) or not responding properly with the insulin production and release (type-II diabetes).

Pancreatic islets or islets of Langerhans, referred to herein as islets, are clusters of cells, containing mostly beta cells that secrete insulin. In people suffering from type-I diabetes, the islets are destroyed. One medical solution is to implant islets. In islet transplantation, cells are isolated from a donor pancreas and transplanted into type I diabetic patients. Once implanted, the transplanted islets begin to make and release insulin, thereby helping patients potentially avoiding the need of daily insulin injections. Furthermore, a potential alternative to the transplantation of islets is the transplantation of stem cell-derived cells, such as stem cell-derived beta cells.

Islet transplantation into the liver of diabetic patients has been studied for decades as a long-term treatment of type-I diabetes by normalizing blood sugar levels and preventing life-threatening hypoglycemic episodes. However, this “intrahepatic” islet transplantation results in chronic decline of islet function due to inflammation, immune response, and toxic environment to islets.

Attempts have been made to transplant islets into sites outside the liver. For example, the subcutaneous site (e.g., under the skin) is promising as it provides a large area and easy access for transplantation. However, low oxygen supply to implanted islets within the subcutaneous microenvironment is detrimental to islet survival. Specifically, the survival of islets depends on sufficient supply of oxygen to the islets at the site of implantation. Inadequate flow of oxygen, and/or of other nutrients, leads to the death of the islets, thereby negating any benefits of the implantation.

For a period of time after a subcutaneous implantation, a risk for ischemia exists. Ischemia is caused by inadequate blood flow due to the lack of adequate vascular structure in the subcutaneous implantation site. Oxygen supply to the implanted islets is not proper until sufficient vascular growth is achieved around the islets. Accordingly, for the islets to survive during a period of time between implantation and vascular growth, oxygen should be adequately supplied from other sources. Some proposed solutions exist currently for the oxygen supply in islet transplantation outside of the liver.

To date, the use of subcutaneous sites has not necessarily demonstrated adequate engraftment efficiency compared to the liver. Therefore, current treatments of diabetes based on islet implantation have several disadvantages that need to be overcome.

### SUMMARY

At least two challenges of subcutaneous islet transplantation have been observed for improving subcutaneous implantation as a site of transplantation other than or in addition to the liver: 1) low oxygen (O2) in the subcutaneous tissue, which can lead to acute islet death or death of stem cell-derived cells; and 2) graft aggregation, which prohibits sufficient O2 diffusion into the grafts thereby exacerbating the low O2 problem. Graft aggregation is a subcutaneous site-specific issue and often overlooked. In the conventional liver site, islets disperse throughout the liver.

Disclosed herein are embodiments of oxygenation of subcutaneous-transplanted islet grafts (or stem cell-derived cells) using O2 inhalation therapy. For local SC oxygenation, there is disclosed a simply structured O2 transporter device configured to transport O2 from an ambient air location into or toward a subcutaneous site. The transporter device includes a component that sandwiches or otherwise encloses or is coupled to an islet graft between two or more layers, such as mesh layers. In an embodiment, the cells are dispersed through an entirety or a portion of the mesh rather than being sandwiched. In another embodiment, the islet graft is coupled to a single layer of mesh. The layers are configured to modify the graft shape, which limits aggregation, prevents hypoxia, and improves engraftment. The system uses or is otherwise coupled to a mesh sheet at the subcutaneous site to prevent aggregation by holding islets in thin layers.

The disclosed transporter device uses an external O2 receiver that is coupled to a cannula, which communicatively connects to one or more multi-layered flexible mesh sheets. The sheets sandwich islet graft as thin layers in a confined area at the subcutaneous site. In a non-limiting example, the mesh comprises interconnecting microcapillaries made of a highly biocompatible and O2-permeable polymer to transport O2. The polymer may be, for example Parylene-HT. In addition, the mesh structure supports physiological graft revascularization for long-term engraftment.

Thus, generally described is a microfabricated, implantable medical transporter device with an ambient or extracorporeal component and a subcutaneous component connected by a cannula in the middle, where the subcutaneous component is configured to retain live cells such as in a state coupled to one or more flexible mesh sheets. At least a portion of the cannula can be impermeable to oxygen. The implantable medical device is used to implant the live cells, such as islets or stem cell-derived cells, in an implantation site, such as a subcutaneous site and provide nutrients to the live cells, thereby enabling their survival. As mentioned, the subcutaneous component comprises one or more multi-layered flexible mesh sheets that sandwich islet graft as thin layers in a confined area.

In a non-limiting example embodiment, one of the components (such as the ambient component) is fully or partially permeable to a predefined class of small molecules of interest, such as diatomic oxygen (O₂) or other “drugs.” In another embodiment, the device does not include the absorption component. The absorption component can also be removably attached to the device such as to the cannula. The small molecules generally provide nutrients to the live cells. This component is referred to herein as an absorption component. Specifically, the permeability of the absorption component enables permeation of the small molecules from a surrounding environment into the absorption component. The other component, referred to as a discharge component, can be partially permeable to the small molecules, where the permeation is at a specific location of the component. The live cells are retained on or within at least a portion of the discharge component, wherein the discharge component at least partially includes one or more layers of mesh sheets that sandwich islet graft as thin layers in a confined area. The small molecules are transported from the absorption component to the discharge component via the cannula and permeates through the permeation area to the live cells.

The components can be sized to collect and disburse an estimated amount of the small molecules and transfer them by passive means, that is, by virtue of there being a higher concentration of the molecules in one region than another region. Proteins to assist in the capture and transport of the target small molecule can also be included within the device.

The cannula can include a tube or strip of pliable, bendable material so that a surgeon can bend the cannula and keep it bent in order to align the device in the body. The material of the cannula can vary and can include, for example, flexible materials such as silicone, polyurethane, and metal. For example, the device can be mounted so that its cannula enters the subcutaneous site and bends back so that the discharge component sits below the skin. Suture holes can be included to assist implantation.

In an embodiment, the implantable medical device includes an absorption component, a cannula, a discharge component. At least a portion of the absorption component is permeable to a predefined class of small molecules, such as molecule oxygen. A first portion of the discharge component is permeable to the small molecules, whereas a second portion (e.g., the remaining portion) of the discharge component when present is impermeable to the small molecules. The cannula includes a lumen. The lumen is impermeable to the small molecules and connects an interior of the absorption component to an interior of the discharge component. The implantable medical device also includes a means for retaining live cells and for providing the small molecules to the live cells based on permeation through the first portion of the discharge component. Permeable and impermeable portions can be defined by using specific materials. Various materials are available and are biocompatible and/or biodegradable. For instance, silicone and Parylene HT is used to define permeable portions. A coating of parylene C can be used to reduce the permeability and, thus, define impermeable portions. Parylene has several subtypes, which are within the scope of this disclosure, and with different permeability, which is also within the scope of this disclosure. Parylene C is O2-impermeable while Parylene HT is O2-permeable. In an embodiment, the absorption component, when present, does not have any portion that is permeable to a predefined class of small molecules, such as molecule oxygen. The absorption component can include a reservoir, such as an oxygen reservoir (e.g., a compressed cylinder, an oxygen-generating material, and/or a hydrolysis chip, or the like) that stores or generates the predefined class of small molecules for transport across the lumen.

In a non-limiting example, the means can include a reservoir. The reservoir can be, for example, one or more multi-layered flexible mesh sheets that sandwich islet graft as thin layers in a confined area. The reservoir is configured to retain live cells to provide the small molecules (e.g., oxygen) to the live cells based on permeation of the small molecules through the discharge component. The discharge component and the absorption component can be dimensioned based on an expected consumption of the small molecules by the live cells. The reservoir can have a cylindrical shape. Its internal diameters is in the range of 1 mm to 20 mm. Its height falls in the range of 100 μm to 1 mm.

In addition, the culture is added to the means and the small molecules (e.g., oxygen) permeates to the culture through the first portion of the discharge component. In an example, the live cells include islets and the culture includes hydrogel. In this example, the hydrogel includes vinyl sulfone and cysteine.

In an embodiment, a method of using an implantable medical device is described. The method includes providing the implantable medical device. The implantable medical device includes an absorption component and a discharge component connected by a cannula. The implantable medical device may also include a reservoir. The reservoir can be, for example, one or more multi-layered flexible mesh sheets that sandwich islet graft as thin layers in a confined area. The method also includes adding live cells to the reservoir. The method also includes placing at least the reservoir retaining the live cells, the discharge component, and a portion of the cannula inside a body of a subject. The method also includes securing the implantable medical device in place.

In an example, the method further includes placing the absorption component at an external surface of a skin of the subject and suturing the absorption component to the skin. Alternatively, the absorption component is placed inside the body of the subject and can be sutured to tissue.

In an example, the live cells include islets. In another example, the cells are stem cell-derived cells such as stem cell-derived beta cells, which can be used in place and/or in conjunction with islets such that the transporter device can be used to transport stem cell-derived cells. The method further includes determining vascular growth around the islets after a period of time and removing the implantable medical device from the body of the subject after the period of time. Similar devices are described in U.S. Pat. No. 10,092,387, which is incorporated herein by reference in its entirety.

A further understanding of the nature and the advantages of the embodiments disclosed and suggested herein may be realized by reference to the remaining portions of the specification and the attached drawings.

### BRIEF DESCRIPTION OF THE DRAWINGS

FIG. 1 illustrates an example of an implantable medical device, in accordance with an embodiment.

FIG. 2 illustrates a multi-layered mesh of the implantable medical device including an enlarged view of a portion of the mesh.

FIG. 3 illustrates another, alternate example of an implantable medical device.

### DETAILED DESCRIPTION

Implantable medical devices, their methods of manufacture, and methods for their use are described. The implantable medical devices facilitate implanting live tissues in the body and providing nutrients to the live tissues for their survival. The implantable medical devices capture the nutrients from an environment external to the body and/or from within the body and deliver the nutrients to the live tissues.

Embodiments of the present disclosure include an implantable medical device that facilitates implantation of live cells in a body of a subject and targeted supply of small molecules to the live cells for their survival. Specifically, the implantable medical device includes, among other components, a discharge component. The discharge component has a particular portion permeable to the small molecules. The live cells are retained at a location coupled to the discharge component wherein such a location can comprise one or more multi-layered mesh sheets that sandwich or are otherwise coupled to an islet graft as thin layers in a confined area. The small molecules are supplied to the live cells in part through the permeation from the particular portion of the discharge component. Prior to the implantation, a determination may be made as to the desired amount of the live cells. The consumption of the small molecules by such an amount can be estimated. The estimated consumption can be correlated to a particular size and/or permeation of the implantable medical device such that the appropriate implantable medical device can be obtained and implanted.

In an embodiment, an implantable medical device is used to implant islets in a subcutaneous site. The implantable medical device is secured in place for a period of time. A reservoir of the implantable medical device can retains the islets and is placed in the subcutaneous site through an incision. Based on a natural concentration gradient of oxygen, the implantable medical device transports oxygen from an oxygen-rich zone into the subcutaneous site, which is an oxygen deficient zone. The transported oxygen is permeated to the islets, thereby providing adequate oxygen flow for their survivals. Over time, vascular growth is achieved around the islets, thereby creating another source of oxygen. When the vascular growth is sufficient for the survival of the islets, the implantable medical device may be removed, whereas the islets may remain in the subcutaneous site.

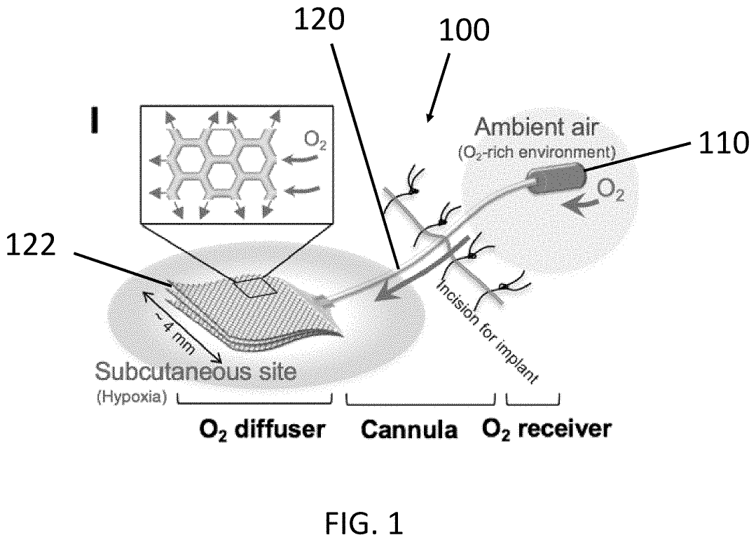

FIG. 1 illustrates an example of a device 100 in accordance with an embodiment. The device 100 includes an absorption component 110 (also referred to as an O2 receiver), a cannula 120, and a discharge component 122 (also referred to as an O2 diffuser or mesh component) configured to be implanted in a subcutaneous location while the absorption component is not subcutaneous. The cannula can extend through or across the skin of a patient. The discharge component includes one or more multi-layered, flexible mesh sheets. The mesh sheets sandwich islet graft as thin layers in a confined area. The absorption component 110 is positioned on the first or proximal end of the cannula 120. The discharge component 122 including the mesh sheets is positioned on a second or distal end of the cannula 120.

The absorption component 110 serves as an external O2 receiver and the cannula 120 connects the O2 receiver to the multi-layered flexible mesh sheets that sandwich the islet graft as thin layers in the confined area. In an embodiment, as mentioned, the mesh discharge component 122 is configured as a mesh sheet or a collection of interlayered sheets. Each mesh sheet is configured to prevent aggregation by holding islets in thin layers. The mesh sheets provide the device with a compact shape. In an embodiment, the mesh discharge component is removable from the cannula such that the mesh discharge component permits permanent wound closure.

In a nonlimiting example the mesh of the mesh sheets comprises two or more interconnecting microcapillary elements made of a highly biocompatible and O2-permeable polymer (such as Parylene-HT) to transport O2. The mesh can be made of other materials including Polyether ether ketone (PEEK) and biodegradable material(s) including poly(lactic-co-glycolic acid) (PLGA). In addition, the mesh structure supports physiological graft revascularization for long-term engraftment. In a nonlimiting example, the mesh has a thickness of less than 0.1 mm. In another example, the mesh comprises at least one thin wall mesh having a thickness of 100 to 5 μm. An example advantage of mesh is that it supports physiological graft revascularization. The material such as Parylene can form a membrane and can be sufficiently thin to achieve a desired permeability relative to a predetermined molecule such as to achieve a desired oxygen permeation.

A desired functioning of the device may transport sufficient oxygen from a source to the cells, where oxygen is heavily consumed. The total oxygen consumption rate (OCR) is related to the metabolism and the volume of transplanted cells. In an example, this can be in the non-limiting range of 1×10⁻¹⁴ to 1×10⁻¹² [mol/s].

Based on such an amount of oxygen needed per time, the discharge component can have sufficient oxygen permeation flux (J) across a barrier membrane of the device, such as a barrier membrane of the mesh or other portion of the device. The permeation flux can be defined as

J=P·(Δp/l) [mol/s]

where P is the gas permeability of the membrane, Δp is the applied pressure difference across the membrane, l is the thickness of the membrane. The gas permeability of membrane is then expressed as:

P=D·S[(mol·m)/(m²·s·Pa)]

where D is the Fick's diffusion coefficient [m²/s] and S [(s²·mol)/(kg·m²)] is the solubility coefficient of membrane.

The permeation of oxygen across a polymeric membrane is a complex and multi-step process. A gas permeability across the membrane can depend on several factors including temperature, porosity, crystallinity, and thickness. To achieve a desired total oxygen flux (J) (such as a maximum flux), a thinly deposited parylene film (order of submicron to micron) can be used. As such, not only the oxygen gradient across membrane can be increased, the intrinsic oxygen permeability of parylene can also be increased (compared to bulk parylene).

To have permeation flux (J) greater than total oxygen consumption rate (OCR), the permeability of a chosen barrier material is at least 5×10⁻⁶ [Barrer] (where Barrer=10-10 [(cm3 (STP)·cm)/(cm2·s·cmHg])≅3.35×10-10 [(mol·m)/(m2·s·Pa)) in a non-limiting example. Comparable calculation could also be performed to accommodate different types of cell transplantation where different discharge components may be required.

Pursuant to a method of manufacture of the mesh, the mesh can be an interconnected or interwoven network of sacrificial material, such as for example, a metal, sugar or a light-sensitive material, that is used as a template for the capillary elements of the mesh. The sacrificial material can be conformably coated with a thin layer of a gas permeable polymer, such as 1 um Parylene HT, which can serve as one or more walls to a capillary lumen. The sacrificial materials are leached out thereby leaving the highly interconnected, hollow network of capillaries. The hollow network allows for efficient transfer of gas throughout the graft via diffusion and down the capillaries permeation out of the polymer surface. This configuration allows for large grafts to be supported via diffusion.

In an embodiment, the network of capillary elements of the mesh functions at least to average tension of oxygen over a volume of the entire mesh. Implantation of a cell graft within the mesh effectuates oxygenation of interior cells by siphoning oxygen from a peripheral region of the mesh.

The interconnectedness of the capillary network formed by the mesh provides redundant pathways for gas to transport throughout the graft, making it resilient to defects or damage. The hydrophobicity of the luminal surface of the capillaries prevents infiltration of fluid into the network. Patterning of appropriate capillary diameter and wall thickness prevents buckling or collapse following implantation. Patterning of capillary spacing and cross section ensures sufficient oxygen supply to graft. The conformal coating process creates an implant with minimal material and high biocompatibility when produced with Parylene (USP Class VI).

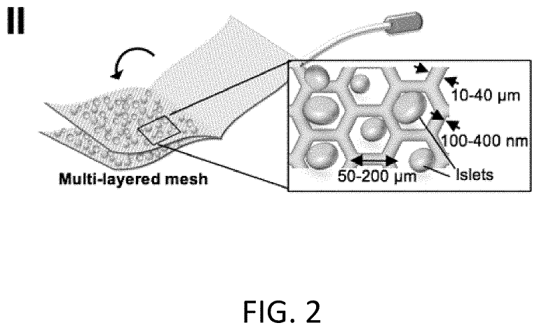

FIG. 2 shows a non-limiting example of a multilayered mesh structure and includes an enlarged view of a portion of the mesh. As shown, the mesh comprises a series of interconnected pores or cells formed by capillary structures. Each of the capillary structures can define internal capillary lumens. The cells provide locations for islets. In a nonlimiting example, each of the capillary structures has a thickness or diameter of about 10 to 40 μm with each of the capillary structures being formed of walls having a thickness of about 100 to 400 nm. It should be appreciated that the dimensions of the capillary structures can vary. For example, the mesh size and a hollow channel size can be 1-500 μm and the capillary structures being formed of walls can have a thickness of about to be 0.1-100 μm.

### Alternate Example

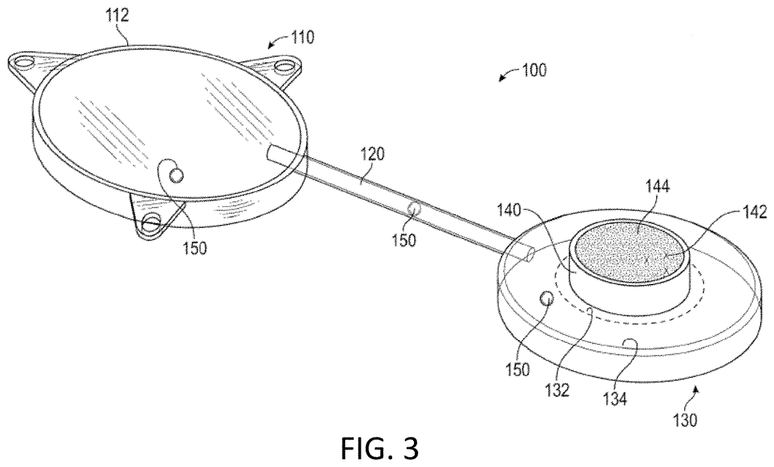

FIG. 3 shows an alternate example of a device 100 having a discharge component 130, and a means 140 for retaining live cells 142 and providing small molecules 150 to the live cells 142. The device shown in FIG. 3 can interchangeably share one or more components with the device shown in FIG. 1. In an example, the live cells 142 are islets including pancreatic beta cells and the small molecules 150 are molecular oxygen (O₂). The small molecules 150 are passively captured by the absorption component 110 and transported to the discharge component 130 through the cannula 120. The small molecules 150 are then permeated into the means 140 for consumption by the live cells 142.

In an example, the absorption component 110 is partially or fully permeable to the small molecules 150. For instance, the entire membrane that forms the absorption component 110 or only a portion of the membrane is permeable to the small molecules 150. The small molecules 150 permeates to an interior of the absorption component 150 through the permeable membrane or permeable portion thereof. The absorption component 110 may also be foldable, rollable, and/or stretchable depending on the membrane.

The cannula 120 includes a thin lumen that connects the interior of the absorption component 110 to an interior of the discharge component 130. The cannula 120 is impermeable to the small molecules 150 such that the small molecules 150 are transported between the two interiors through the lumen and without permeation at the cannula 120. For example, the cannula 120 is formed by a membrane coated with a material that renders the cannula 120 impermeable to the small molecules 150. Based on natural concentration gradient of the small molecules 150, transportation occurs from the absorption component 110 to the discharge component 130. That is the case when the absorption component 110 is placed in a region that has a higher concentration of the small molecules 150 relative to the concentration in a region where the discharge component 130 is placed.

The discharge component 130 includes a particular portion 132 (e.g., a first portion) that is permeable to the small molecules 150. The remaining portion 134 of the discharge component 130 (e.g., a second portion) is generally impermeable such the permeation of the small molecules 150 is targeted to occur through the particular portion 132. The discharge component 130 may also be foldable, rollable, and/or stretchable depending on membrane that forms the discharge component 130.

The means 140 is external to the discharge component 130, retains the live cells 142, and supplies the small molecules 150 to the live cells 142 based on the permeation from the permeable portion 132 of the discharge component 130. Different types of the means exist including, for instance, a reservoir, irregular array of corrugations, or an adhesion. Generally, the live cells 142 belong or are included in a culture 144 retained by the means 140. The culture 144 represent a solution in which the live cells can be placed and that provides a suitable environment for their survivability. A hydrogel is an example of the culture 144. The supply of the small molecules 150 to the live cells 142 can be targeted by properly locating the means 140 relative to the permeable portion 132 of the discharge component 130. For example, the means 140 is placed on top of the permeable portion 132 and has a bottom surface that is formed by the permeable portion 132, that surrounds the permeable portion 132, or that is approximately surrounded by the permeable portion 132 (e.g., the permeable portion surrounds the bottom surface by a margin that does not exceed 10% (or some other relevant percentage) the total area of the bottom surface)).

Various materials are available and are biocompatible and/or biodegradable. In an example, the absorption component 110, the cannula 120, the discharge component 130, and the means 140 are made of biocompatible silicone that has been cured together, i.e., integrally formed. Parylene C coating surrounds cannula 120, the remaining portion 134 of the discharge component 130 (but not the permeable portion 132 when present), and, optionally, a portion (but not the entire) absorption component 110. Parylene C is a biocompatible polymer with a permeability rate that is five orders of magnitude lower than silicone. The coating renders the coated portions impermeable to the small molecules 150. In an embodiment, the cannula 120 is made of PEEK although the material of the cannula can vary.

“Permeability” of a material is typically in relation to a size of substance of interest. A Stokes-Einstein radius or a Stokes diameter is a measure of the diffusion properties of a substance. A “Stokes diameter” is an equivalent diameter of a hard sphere that a molecule possesses in terms of its diffusion rate. A molecule can pass through thin materials with pores that have a Stokes diameter that is about 1 to about 5 times the Stokes diameter of the molecule.

“About” includes within a tolerance of ±0.01%, ±0.1%, ±1%, ±2%, ±3%, ±4%, ±5%, ±8%, ±10%, ±15%, ±20%, ±25%, or as otherwise known in the art.

The small molecules 150 diffusion out of the discharge component 130 into the means 140 lowers the device's 100 internal concentration, and this in turn pulls additional small molecules from a small molecule rich region (e.g., where the absorption component 110 is located) into the device 100. The concentration gradient will continue to transport small molecules from the rich region into the means 140, thereby providing an adequate flow of the small molecules to the live cells 142.

Dosing and targeted release can be controlled by material properties of the device 100. Controlling the thickness of silicone can determine the permeation rate (dosing). As the absorption component 110, cannula 120, and discharge component 130 are integrally formed with the same thickness of silicone, a single adjustment to how much silicone is distributed on a mold can determine permeation rates. Applying impermeable coating to specific portions of the device 100 allows control over the permeation rates and/or locations of the permeations.

The dimensions of the absorption component 110 and discharge component 130 can also be adjusted to alter the permeation rate. Generally, the larger the permeable surface area, the larger the permeation rate is (given a same concentration of small molecules). The dimensions and permeable surface areas are application dependent and can be configured for the specific task the device 100 is to perform. For instance, a desired amount of live cells 142 can be determined. The means 140 is dimensioned to hold that amount. An estimated consumption of the small molecules 150 by the amount of the live cells 142 is estimated. The dimensions and permeable surface areas of the absorption component 110 and discharge component 130 are set to provide a flow of the small molecules 150 adequate for the estimated consumption. The device 100 is manufactured accordingly.

In addition to controlling the thickness, one may inject into the interior of the device 100 a substance with a high diffusion constant such as perfluorocarbons, air, etc. For example, a perfluorocarbon within the absorption component 110 and device 100 can increase oxygen solubility (e.g., in the case when the small molecules 150 are oxygen). A hemeprotein, such as a natural, artificial, or autologous hemoglobin or myoglobin, can be added inside the device 100 to increase oxygen transport. A chlorocruorin or a hemocyanin can be added into the absorption component 110 and other portions of the device 100 to increase oxygen transport. Other substances natural or synthetic that have beneficial properties for small molecule storage or transport may be used.

Other small molecules besides diatomic oxygen can also be captured and transported. The device 100 can be targeted for carbon dioxide (CO₂), nitrous oxide (N₂O), or other gases. Small molecule proteins and other drugs can be specifically targeted. Any of these ‘drugs’ may be transported, whether they are classified as a therapeutic agent, waste product, or otherwise.

An example method of use, in accordance with an embodiment, is now described. In a first step, a device is provided, where the device includes an absorption component, a cannula, a discharge component, and an optional reservoir. The cannula includes a tube and connects the absorption component and the discharge component. The reservoir is external to the discharge component and is attached to a surface of the discharge component.

In a next step, live cells are added to the reservoir of the device. For example, parts of vinyl sulfone (VS) functionalized saccharide-peptide copolymer and cysteine (Cys) functionalized saccharide-peptide copolymer are mixed to form a culture. The live cells are added to the culture. The culture is then moved to a syringe. The syringe is used to add the culture to the reservoir.

In a next step, an incision is cut in a body of a subject. For example, a ten millimeter incision is cut into the abdomen of the subject. The cannula with the pliable metal tube is then bent into position.

In a next step, at least the reservoir retaining the live cells, the discharge component, and a portion of the cannula are placed inside the body. In an example, the reservoir, discharge component, and portion of the cannula are pulled through the incision. For instance, these components of the device are placed in a subcutaneous site in the abdomen area.

In a next step, the device is secured in place. In an example, the absorption component is placed outside of the body. In another example, the absorption component in subcutaneous site, but at a location with relatively higher oxygen concentration (or a relatively higher concentration of other small molecules usable as nutrients for the live cells). In both example, the device can be secured in place by suturing the absorption component to surrounding skin or tissue.

In a next step, vascular growth around the live cells can be determined after a period of time. In an example, the vascular growth can be expected over time. In another example, a probe is used to determine the vascular growth.

In a next step, the device is removed from the body after the period of time. For example, if the vascular growth is satisfactory (e.g., provides adequate oxygen flow or an adequate source of nutrients to the live cells), the device is removed. Removing the device includes removing the absorption component, the cannula, and the discharge component. Optionally, the reservoir is removed. However, the live cells are not removed and are retained in the body. A surgical tool can be used to cut an incision in the body and remove any of the desired components of the device.

An implantable medical device is referred to as a device in the present disclosure. In other words, unless context dictates otherwise, a device as used herein represents a medical device that can be implanted in a body of a subject. The implantation need not be permanent and, instead, can be temporary. The device can be secured in place for the period of the implantation using different techniques, as further described in the next figures.

Embodiments of the present disclosure are described in connection with a device for implanting islets and supplying oxygen to the implanted islets. However, the embodiments are not limited as such. Instead, the device is also usable for implanting other types of live cells and for supplying other types of nutrients to the live cells. Generally, a live cell can be any cell that relies on a nutrient for survival. A nutrient represents a molecule that the cell can consume through cellular metabolism, alone or in combination with other molecules, to survive. Islets are one example of live cells. Oxygen is one example of nutrients.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain. “About” includes within a tolerance of ±0.01%, ±0.1%, ±1%, ±2%, ±3%, ±4%, ±5%, ±8%, ±10%, ±15%, ±20%, ±25%, or as otherwise known in the art. “Substantially” refers to more than 66%, 75%, 80%, 90%, 95%, or, depending on the context within which the term substantially appears, value otherwise as known in the art.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

1. An implantable medical device, comprising:
an absorption component that comprises at least one portion permeable to oxygen and that defines an interior;
a discharge component that comprises a first portion permeable to the oxygen, wherein the discharge component includes one or more flexible mesh sheets, and wherein the mesh sheets are coupled to cells in a confined area; and
a cannula that comprises a lumen, wherein the lumen of the cannula connects the interior of the absorption component to the interior of the discharge component.

2. The implantable medical device of claim 1, wherein the cannula is flexible.

3. The implantable medical device of claim 1, wherein the cannula is at least one of silicone, polyurethane, PEEK, and metal.

4. The implantable medical device of claim 1, wherein each mesh sheet comprises a multi-layered mesh sheet.

5. The implantable medical device of claim 1, wherein each mesh sheet comprises two or more interconnecting microcapillary elements.

6. The implantable medical device of claim 5, wherein each microcapillary element comprises a highly biocompatible and O2-permeable polymer to transport O2.

7. The implantable medical device of claim 6, wherein the polymer is Parylene.

8. The implantable medical device of claim 6, wherein the polymer is Parylene C or Parylene HT.

9. The implantable medical device of claim 1, wherein each mesh sheet comprises an interconnected or interwoven network of sacrificial material.

10. The implantable medical device of claim 9, wherein the sacrificial material is a metal, a sugar, a polymer, or a light-sensitive material.

11. The implantable medical device of claim 1, wherein the cannula is configured to extend through or across skin of a patient while the absorption component is positioned discharge component is in a subcutaneous location of the patient.

12. The implantable medical device of claim 5, wherein each microcapillary element has a wall thickness of 0.1-100 μm.

13. The implantable medical device of claim 1, wherein the cells comprise at least one of an islet graft and a stem cell-derived cell.

14. The implantable medical device of claim 1, wherein the mesh sheets sandwich the cells as thin layers.

15. The implantable medical device of claim 1, wherein the cells are dispersed throughout at least a portion of the mesh.

16. The implantable medical device of claim 1, wherein the discharge components is configured to be placed at a subcutaneous site.

17. The implantable medical device of claim 1, wherein at least a portion of the cannula is impermeable to the oxygen.

18. A method of using an implantable medical device, the method comprising:
providing an implantable medical device, wherein the implantable medical device comprises:
an absorption component that comprises at least one portion permeable to oxygen and that defines an interior;
a discharge component that comprises a first portion permeable to the oxygen, wherein the discharge component includes one or more flexible mesh sheets, and wherein the mesh sheets are coupled to cells in a confined area;
a cannula that comprises a lumen, wherein the lumen of the cannula connects the interior of the absorption component to the interior of the discharge component;
placing at least a portion of the cannula and the discharge component inside a location of body of a subject; and
securing the implantable medical device in place.

19. The method of using the implantable medical device of claim 18, wherein the location of the body of the patient is a subcutaneous location.

20. The method of using the implantable medical device of claim 18, wherein the cannula is flexible.

21. The method of using the implantable medical device of claim 18, wherein the cannula is at least one of silicone, polyurethane, and metal.

22. The method of using the implantable medical device of claim 18, wherein each mesh sheet comprises a multi-layered mesh sheet.

23. The method of using the implantable medical device of claim 18, wherein each mesh sheet comprises two or more interconnecting microcapillary elements.

24. The method of using the implantable medical device of claim 18, wherein the cells comprise at least one of an islet graft and a stem cell-derived cell.

25. The method of using the implantable medical device of claim 18, wherein the mesh sheets sandwich the cells as thin layers.
